(19) United States
(12) Patent Application Publication
(10) Pub. No.: US 2018/0093069 A1
(43) Pub. Date: Apr. 5, 2018

(54) ASYMMETRIC CATHETER TIPS

(71) Applicant: CATHAWAY MEDICAL (2012) LTD., RAMAT GAN (IL)

(72) Inventors: Eyal BRESSLER, RAMAT GAN (IL); Eran EILAT, HERZLIYA (IL)

(73) Assignee: CATHAWAY MEDICAL (2012) LTD., RAMAT GAN (IL)

(21) Appl. No.: 15/723,211

(22) Filed: Oct. 3, 2017

Related U.S. Application Data
(60) Provisional application No. 62/404,230, filed on Oct. 5, 2016.

(51) Int. Cl. A61M 25/00; A61M 25/10

(52) U.S. Cl. CPC A61M 25/007; A61M 2210/1085; A61M 25/10

(57) ABSTRACT

The present invention discloses a catheter tip having a main longitudinal axis with a tapered distal portion having an enveloping external wall defining an inner space; wherein the thickness of said wall is radially asymmetric with respect to said axis. The present invention also discloses a catheter tip having a main longitudinal axis having a tapered distal portion comprising an enveloping external wall defining an inner space; wherein said tip is provided with at least one first small aperture and at least one second large aperture, said apertures are located approximately opposite each other. The present invention further discloses a catheter tip having a main longitudinal axis with a tapered distal portion having an enveloping external wall defining an inner space; wherein said tip is provided with at least one first small aperture and at least one second large aperture, said apertures are located approximately opposite each other; and wherein said apertures are cutouts; jambs of said cutouts are tilted from the outer edge to the inner edge away from the perpendicular.

### CROSS REFERENCE TO RELATED APPLICATIONS

This application is a U.S. National Application under 35 U.S.C. 111(a), claiming priority from U.S. Provisional Application No.: 62/404,230, filed 5 Oct. 2016, hereby incorporated by reference in its entirety.

### FIELD OF INVENTION

The present invention generally pertains to asymmetric catheter tips and to production and catheterizing methods thereof.

### BACKGROUND OF THE INVENTION

A urinary catheter is characterized by a tubular shaft extending between an insertable end and a discharge end, and a tip fixedly connected to the insertable end of the tubular shaft. Nyman et al. underlines that insertion of a urinary catheter into the urethra is often cumbersome, and associated with certain risks, see for example US2016243331 and US2005192560. Nyman et al. underlines that insertion of a urinary catheter into the urethra is often cumbersome, and associated with certain risks, see Nyman et al., US2016184551 by Dentsply (US) which is incorporated herein as a reference. Nyman et al. further states that this is particularly the case for male catheters. A male catheter is relatively long, typically 35-40 cm, to be able to extend through the whole length of the urethra. The insertable length of the catheter is normally at least 200-350 mm for male users. The male urethra is also curved in many places, and comprises sections with reduced cross-sections.

The hardness of the tip is reviewed in the literature. As indicated by Nyman et al., during the introduction of the catheter into the urethra and while guiding the catheter tip through the urethra into the bladder, it is necessary to overcome pockets, folds, bends, strictures, and/or the like. If one pushes the catheter with a corresponding force against the existing impediments in the urethra, one will face a considerable risk of injury.

To overcome tip-shape and hardness-related insertion problems, it is known to use special tips on the catheter. For example, the catheter may be provided with a curved tip, often referred to as a Tiemann or Coudé type catheter. Ruesch et al, EP0384476 which is incorporated herein as a reference, provides catheters with a flexible or elastic tip which is conical. Sauer, U.S. Pat. No. 7,717,902, discloses a catheter having an enlarged, ball-shaped tip, having a diameter widely exceeding the diameter of the rest of the catheter shaft. US2014378951, U.S. Pat. No. 5,919,170 which are incorporated herein as a reference and others disclose an elongated catheter that has a main longitudinal axis with a substantially circular cross section: rounded cross section or polygonally shaped cross section, as disclosed in WO15090338. A tip, located at its insertable distal portion, includes a few (e.g., two) drainage apertures (also called eyelets). In some arrangements, both apertures are located at 12 o'clock. In other arrangements, the two or more apertures at not located at the same angle: e.g., a first aperture is located at 12 o'clock whilst the second located at 6 o'clock etc.

U.S. Pat. No. 5,549,552 discloses a balloon dilation catheter with improved pushability by improving the shape of the balloon. U.S. Pat. No. 6,113,579 discloses catheter tip designs and methods for improved stent crossing. Both patents do not show safe means and methods for increasing pushability, trackability and crossability of the catheters by new designs of the catheter tip, whereas those three parameters are discussed in literature without referring to the required new tip design, See Schmidt, Wolfram, et al. “A comparison of the mechanical performance characteristics of seven drug-eluting stent systems.” Catheterization and Cardiovascular Interventions 73.3 (2009): 350-360 which is incorporated herein as a reference. U.S. Pat. No. 6,113,579 discloses catheter tip designs and methods for improved stent crossing, yet this patent does not aimed or useful for urological catheters.

A safe increase of catheters' pushability, trackability and crossability in human narrow cavities by new designs of catheter tip is hence still a long felt need.

### BRIEF DESCRIPTION OF THE FIGURES

The invention will be better understood upon reading the following description, given purely by way of example and with reference to the accompanying drawings, in which:

FIG. 1 is a schematic cross section of a catheter tip known in the art;

FIG. 2 is a schematic cross section of one non-limiting embodiment of a catheter tip according to the present invention;

FIGS. 3A and 3B are schematic cross sections of a second non-limiting embodiment of a catheter tip with a radially asymmetric wall according to the present invention, in expanded and collapsed configurations, respectively, illustrating how the tip can be connected to an elongated elastomeric shaft;

FIG. 4A is a schematic cross section of a third non-limiting embodiment of a catheter tip according to the present invention, in which the catheter tip has a radially asymmetric wall;

FIG. 4B is a schematic cross section of a fourth non-limiting embodiment of a catheter tip according to the present invention, in which the catheter tip has a radially symmetric wall;

FIG. 4C is a schematic cross section of the catheter tip illustrated in FIG. 4A when the tip is in a collapsed configuration;

FIG. 5 is a schematic cross section of a fifth non-limiting embodiment of a catheter tip according to the present invention illustrating one non-limiting aperture configuration;

FIG. 6 is a schematic cross section of a sixth non-limiting embodiment of a catheter tip according to the present invention illustrating a second non-limiting aperture configuration;

FIG. 7 is a schematic cross section of a seventh non-limiting embodiment of a catheter tip according to the present invention illustrating a third non-limiting aperture configuration;

FIG. 8 is a schematic cross section of an eighth non-limiting embodiment of a catheter tip according to the present invention illustrating a fourth non-limiting aperture configuration;

FIG. 9 is a schematic illustration of one non-limiting embodiment of a catheter according to the present invention in which the asymmetric tip is attached to a shaft;

FIG. 10 is a schematic illustration of one non-limiting embodiment of a catheter according to the present invention in which the catheter comprises a balloon and a bore for inflating the balloon;

FIGS. 11A and 11B are in-scale three-dimensional views of an asymmetric catheter tip according to one non-limiting embodiment of the present invention, in which FIG. 11A is a cutaway view and FIG. 11B is a view of the catheter tip as assembled;

FIGS. 12A and 12B are two cross sectional scale drawings of one non-limiting embodiment of an asymmetric catheter tip according to the present invention;

FIGS. 12C-12G are three-dimensional illustrations (to scale) from five different views of the catheter tip of which cross-sectional views are presented in FIGS. 12A and 12B;

FIG. 13 is a schematic cross section of one non-limiting embodiment of an asymmetric catheter tip according to the present invention, characterized by a hollow interior and no apertures;

FIG. 14 is a schematic cross section of one non-limiting embodiment of an asymmetric catheter tip according to the present invention having no apertures and made of two materials, an exterior wall material and an interior wall material characterized by different properties than the exterior wall material;

FIG. 15 is a schematic cross section of one non-limiting embodiment of an asymmetric catheter tip according to the present invention, characterized by a hollow interior, no apertures, and a completely or almost completely flat distal end;

FIG. 16 is a schematic cross section of one non-limiting embodiment of an asymmetric catheter tip according to the present invention made of two materials, an exterior wall material and an interior wall material characterized by different properties than the exterior wall material, characterized by a completely or almost completely flat distal end;

FIG. 17 is a schematic cross section of one non-limiting embodiment of an asymmetric catheter tip according to the present invention comprising an asymmetric inner envelope displaced from the main axis of the tip;

FIG. 18 is a schematic cross section of one non-limiting embodiment of an asymmetric catheter tip according to the present invention comprising an asymmetric inner envelope displaced from the main axis of the tip in which the inner envelope is itself asymmetric;

FIG. 19 is a schematic cross section of one non-limiting embodiment of an asymmetric catheter tip according to the present invention comprising an asymmetric inner envelope in which the inner envelope forms a coil-like air-filled inner section; and,

FIG. 20 is a schematic cross section of one non-limiting embodiment of an asymmetric catheter tip according to the present invention characterized by an asymmetric outer envelope.

### SUMMARY OF THE INVENTION

It is one object of the invention to disclose a catheter tip having a main longitudinal axis with a tapered distal portion having an enveloping external wall defining an inner space; wherein the thickness of the wall is radially asymmetric with respect to the axis.

It is another object of the invention to disclose a catheter tip having a main longitudinal axis having a tapered distal portion comprising an enveloping external wall defining an inner space; wherein the tip is provided with at least one first small aperture and at least one second large aperture, the apertures are located approximately opposite each other.

It is another object of the invention to disclose a catheter tip having a main longitudinal axis having a tapered distal portion comprising an enveloping external wall defining an inner space and further wherein the tip is provided with at least one first small aperture and at least one second large aperture, the apertures are located approximately opposite each other.

It is another object of the invention to disclose a catheter tip having a main longitudinal axis with a tapered distal portion having an enveloping external wall defining an inner space; wherein the tip is provided with at least one first small aperture and at least one second large aperture, the apertures are located approximately opposite each other; and wherein the apertures are cutouts; jambs of the cutouts are tilted from the outer edge to the inner edge away from the perpendicular.

It is another object of the invention to disclose a catheter tip as disclosed in any of the above, wherein the thickness of the wall is radially asymmetric with respect to the axis.

It is another object of the invention to disclose a catheter tip as disclosed in any of the above, wherein the tip is provided with at least one first small aperture and at least one second large aperture, the apertures are located approximately opposite each other.

It is another object of the invention to disclose a catheter comprising a tip as defined in any of the above.

### DETAILED DESCRIPTION OF THE PREFERRED EMBODIMENTS

The present invention inter alia discloses a catheter tip having a main axis A:A comprising a tapered distal portion characterized by at least one of an outer envelope and an inner envelope being radially asymmetric with respect to main axis A:A. The present invention inter alia also discloses a catheter tip having a main longitudinal main axis A:A comprising a tapered distal portion characterized by a radially asymmetric moment of inertia. The present invention inter alia further discloses tips as defined in any of the above, a wherein a wall thickness is radially asymmetric; wherein an aperture size is radially asymmetric; wherein an aperture edge is at a non-perpendicular angle to the main axis A:A; wherein the catheter tip comprises at least two materials; wherein the at least two materials differ in at least one material property; and/or wherein the at least one material property is selected from a group consisting of hardness, Young's modulus, Poisson's ratio and any combination thereof.

The term ‘about’ refers hereinafter to a value being more than or less than up to 25% of the defined measure. The term ‘wall’ refers hereinafter to the material forming the tip. The terms ‘envelope’ and ‘external envelope’ refer hereinafter to the shape of the exterior of the tip, which is also the shape of the exterior of the wall. The term ‘internal envelope’ refers hereinafter to the shape of the interior of the tip which is also the shape of the interior of the wall. The term ‘wall thickness’ refers hereinafter to the thickness of the wall. The wall thickness at a particular point is the difference between the radius of the envelope at that point and the radius of the internal envelope at that point. The term ‘catheter’ refers hereinafter to any catheter of insertable tube, including a urological or indwelling catheter (e.g., a Foley catheter), cardiac catheters, and any other catheters configured to be inserted in the human body via a narrow boy cavity.

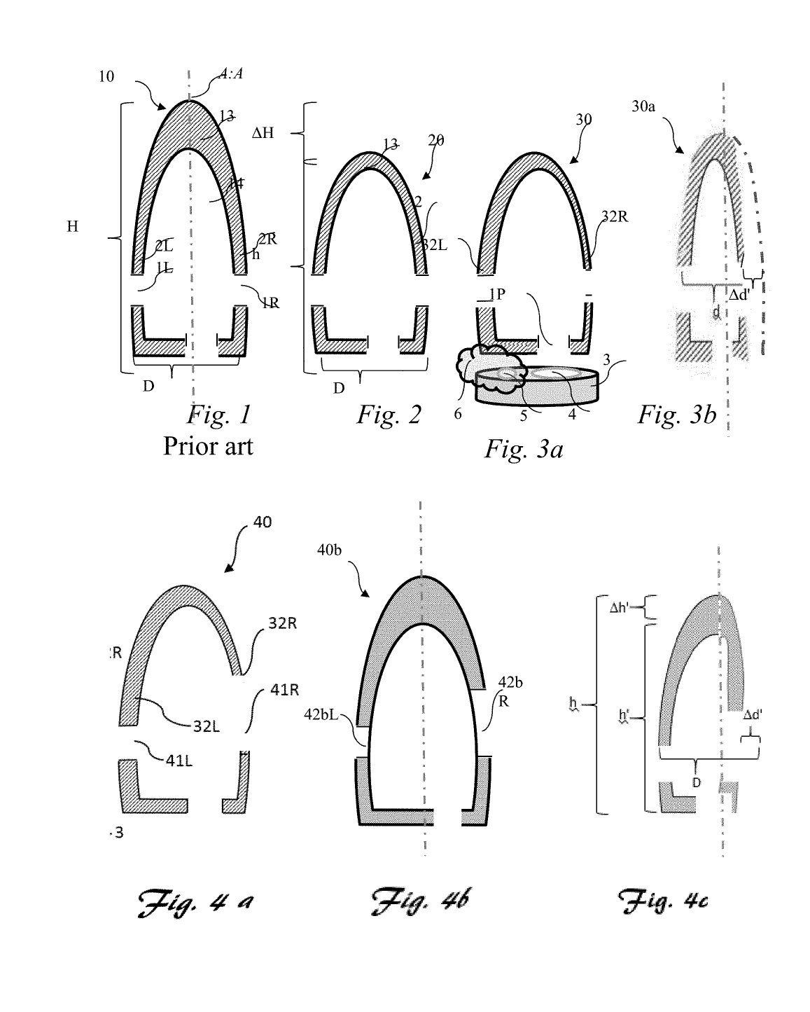

FIG. 1 discloses a prior art urinary catheter tip (10) characterized by a tubular shaft having a main longitudinal axis (A:A) extending between an insertable end and a discharge end (13). This commercially available tip comprises one or more apertures (also known as orifices, openings, draining holes etc.), usually two urine-draining apertures, here a first aperture disposed at 9 o'clock (1L) and a second at 3 o'clock.

Reference is now made to FIGS. 2-8 that highly schematically illustrate a cross section of a tip according to a few embodiments of the invention (e.g., tips 20-50). The distal portion of the tips is fixedly connectable with an elongated open bore(s) shank (3, See FIG. 3). One of the shank's bores (4), being either parallel to or coiled-around the shank axis A:A is in a free fluid (e.g., urine) communication with the tip's proximal aperture (1P). The shank may comprise additional bores, such as a balloon (6) inflating bore (5), where the balloon is accommodatable within the urine bladder and the bore is being either provided in parallel-to or coiled-around shank axis A:A.

Commercially available tips (10) are characterized, inter alia by height H, e.g., about 5 to about 15 mm and distal diameter D (e.g., about 7 to about 17 French, about 2.2 mm to about 5.4 mm). These hollow tips are further characterized by an envelope made of medical grade materials, including silicone rubber, nylon, polyurethane, polyethylene terephthalate (PET), latex, and thermoplastic elastomers and combinations thereof (hereinafter ‘elastomers’), having radially symmetric walls (2L and 2R) ranging from e.g., about 0.75 mm to about 1.50 mm and a radially symmetric dome; e.g., dome maximal height (Hₘₐₓ) ranging from about 11 mm to about 13 mm, i.e., D/DHₘₐₓ ranges between about 0.2 and about 0.5. The tip's maximal height to tip's distal diameter ratio is about 0.3.

The tip height is relevant to both its functionality and safety. A too long (too great a height) tip causes continuous contact between the wall of the urinary bladder and the catheter tip, thereby causing and increasing infection and traumatization of the tissues (See U.S. Pat. No. 4,575,371). Too short a tip decreases its crossability via narrow body lumens. It is hence one aspect of the invention to present a tip of an optimal length (height, h), shorter than commercially available products so it's less traumatic to tissues, yet long enough to be characterized by an effective pushability, trackability and crossability, where H−h=□H. In these embodiments of the invention, □H has a significant measure; hence, the present invention discloses a novel tip with a maximal tip height (h) to tip distal diameter (D) ratio ranging from about 0.15 to about 0.20, namely up to 33% lower than the current ratio for known catheters.

Another aspect of the invention is to disclose a catheter (30) with a radially asymmetric wall according some embodiments of the invention, as schematically illustrated in an out-of-scale manner in FIGS. 3a-b. Radial asymmetry is defined here as an absence of symmetry about an A:A axis or a plane thereof. FIG. 3a shows that the proximal end of the tip is connectable with an elongated elastomeric shaft (3). Shaft 3 may comprise various modules, such as a urine drainage main bore (4), interconnected with the tip's proximal orifice (1P), a fixation balloon (6) provided in a fluid communication with a smaller bore (5) etc. The novelty in this embodiment of the invention is that one or more portions of the envelope (see e.g., 9 o'clock section, 32L) is thicker than other portion(s) of the envelope (see e.g., opposite 3 o'clock section, 32R) so that the moment of inertia of one section (e.g. 32L) is greater than the moment of inertia of at least one other section (e.g., 32R), even though the envelope of the tip is symmetrical. Reference is now made to FIG. 3b. When pushed along via narrow body cavities, tip 30 reversibly collapses from initial diameter D to collapsed diameter d. The narrower (see □d′) collapsed tip has a unique Poisson's ratio (as compared with a regular catheter known in the art) is characterized by enhanced crossability. FIG. 3b illustrates a collapsed tip of a substantially radial symmetrical features.

Those figures hence describe one embodiment of the invention, namely a catheter tip (e.g., 20) having a main longitudinal axis (A:A) with a tapered distal portion (e.g., 13) having an enveloping external wall (e.g., 2) defining an inner space (e.g., 14); wherein the thickness of the wall is radially asymmetric with respect to the axis (e.g., thickness difference as shown from 32L to 32R).

Those figures also describe another embodiment of the invention, namely a catheter tip having a main longitudinal axis having a tapered distal portion comprising an enveloping external wall defining an inner space; wherein the tip is provided with at least one first small aperture (e.g., 42bL) and at least one second large aperture (e.g., 42bR), the apertures are located approximately opposite each other.

Those figures further describe another embodiment of the invention, namely a catheter tip having a main longitudinal axis having a tapered distal portion comprising an enveloping external wall defining an inner space; wherein the tip is provided with at least one first small aperture (e.g., 42bL) and at least one second large aperture (e.g., 42bR), the apertures are located approximately opposite each other; and further wherein the tip is provided with at least one first small aperture and at least one second large aperture, the apertures are located approximately opposite each other.

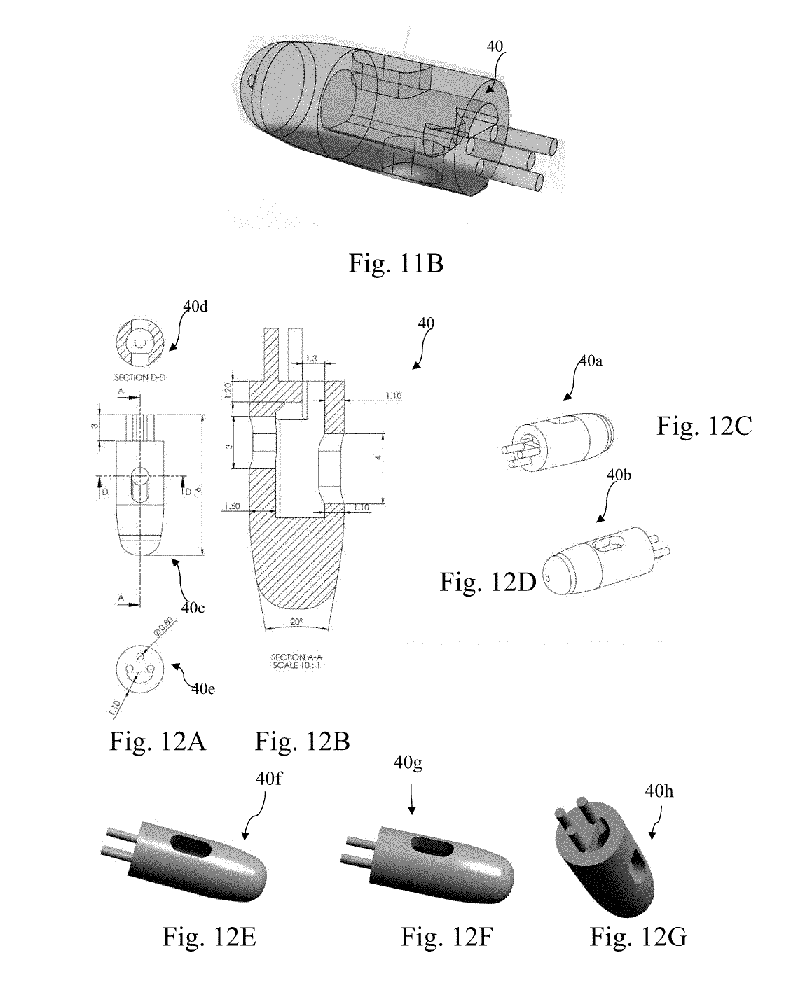

Those figures describe another embodiment of the invention, namely a catheter tip having a main longitudinal axis (A:A) with a tapered distal portion having an enveloping external wall defining an inner space; wherein the tip is provided with at least one first small aperture and at least one second large aperture, the apertures are located approximately opposite each other; and wherein the apertures are cutouts (e.g., 110, FIG. 11); jambs (e.g., 112) of the cutouts are tilted (angle □) from the outer edge to the inner edge (e.g., along axis 111) away from the perpendicular (113).

Those figures also describe another embodiment of the invention, namely a catheter tip as disclosed in any of the above, wherein the thickness of the wall is radially asymmetric with respect to the axis.

Those figures also describe another embodiment of the invention, namely a catheter tip as disclosed in any of the above, wherein the tip is provided with at least one first small aperture and at least one second large aperture, the apertures are located approximately opposite each other.

Those figures also describe another embodiment of the invention, namely a catheter comprising a tip as defined in any of the above embodiments.

It is in the scope of the invention wherein the tip is provided with a bending moment defined as the product between E-modulus and moment of inertia of at least 0.5 MPamm⁴. As noted in US20160015929 which is incorporated herein as a reference, the proximal (insertable) portion of the catheter, for male individuals, must pass prostate in a curved passage, so the catheter's shaft is provided with a bending moment provided in a non-limiting manner e.g., between E-modulus and moment of inertia of less than e.g. about 0.1 to about 0.6 MPamm⁴; See also U.S. Pat. No. 5,746,701, that discloses a guidewire with non-tapered tip and is incorporated herein as a reference.

Another aspect of the invention is to disclose two catheters (40, 40b) with a radially asymmetric wall and a symmetric wall, respectively, according to two embodiments of the invention, as schematically illustrated in an out-of-scale manner in FIGS. 4a-b. In both wall-thick asymmetric tips 40 and 40b, one aperture is significantly bigger than the other: orifice 41L and 42bL at 9 o'clock position is about half as big as opposite orifices 41R and 42bR at 3 o'clock position. Because of the difference in aperture size, for each tip, one portion (e.g., left portion of tip 40 and left portion of tip 40b) has a different amount of material than another portion of the tip (e.g., right portion of tip 40 and right portion of tip 40b) so that the moment of inertia of one portion (e.g. the left portion) is different from the moment of inertia of another portion, while the envelope shape remains substantially the same for the two portions.

A combination of more than one asymmetric feature of the tip is utilizable: i.e., (a) providing the tip with different wall thicknesses across its main longitudinal axis or any of its planes; and (b) providing the orifices with different cross sections, diameter sizes, shapes etc. Hence for example, as schematically shown in FIG. 4c, a profound collapsibility is provided to tip 40b when it pushed along axis A:A, twisted/rotated or pushed-while-rotated around the axis within a body cavity. In contrast to tip 30a which demonstrated a symmetric collapse, tip 40b collapses in asymmetric manner; namely its 3 o'clock side collapses much more than its 9 o'clock side due to both the difference in wall thickness and in that is has apertures of different diameters. Poisson's ratio and rotational-Poisson parameters in tip 40b significantly differ from those of regular tip 10, even in cases where both tips are made of materials with a same Shore-A hardness parameter. The collapse of tip 40b in narrow body cavities, such as the human male urethra adjacent to the prostate, is schematically demonstrated in an out-of-scale manner in FIG. 4c: the tip become longer (See collapsed added length □h′) and narrower (see collapsed reduced diameter □d′). Moreover, its coil-like asymmetric cross-section when collapsed in narrow cavities demonstrates its improved applied Young's modulus E. Young's modulus is calculated by dividing the tensile stress by the extensional strain, in the elastic (initial, linear) portion of the physical stress-strain curve. In our unique case, asymmetric properties alter the applied Young's modulus from that of the regular-shaped tip known in the art:

$$E ≡ \frac{σ ( ɛ )}{ɛ} = \frac{F / {A}_{0}}{Δ L / {L}_{0}} = \frac{{FL}_{0}}{{A}_{0} Δ L}$$

Where E is the Young's modulus (modulus of elasticity); F is the force exerted on an object under tension; A₀ is the actual temporary and reversible asymmetric F□d′ (i.e., a function F of reduced diameter when collapsed) cross-sectional area through which the force is asymmetrically and unevenly applied along axis A:A; ΔL is the temporary and reversible asymmetric amount □h′ by which the length of the object changes; and L₀ is the original length of the object.

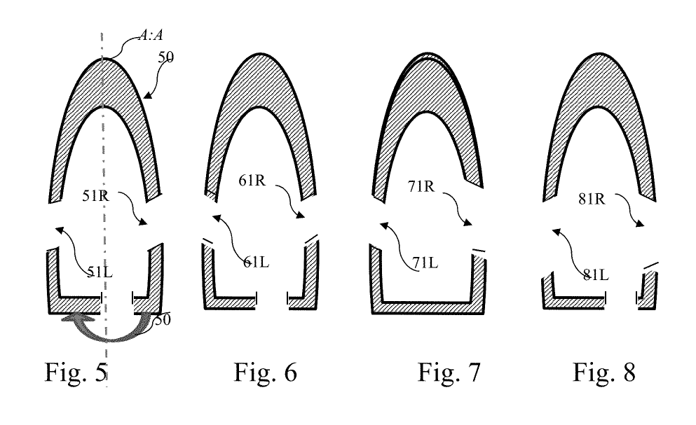

Another aspect of the invention is to disclose various catheters (50) with a radially asymmetric apertures shapes (cross sections) and sizes, according to four embodiments of the invention, as schematically illustrated in an out-of-scale manner in FIGS. 5-8. These examples are provided herein in a non-limiting manner and demonstrate a few of the possible orifice configurations. In all these tip-examples, the orifice edges are not perpendicular to main axis A:A but are tilted at sharp angles to main axis A:A. These tilted orifice edges provide the tip with fine asymmetric properties, especially relevant when catheterization is allowed in a push-and-rotate manner One push-and-rotate example is provided useful when either or both of the main-bore (4) and the smaller-bore (5) are at least partially helically coiled or spirally wounded around main axis A:A. In these cases, there is a synergistic effect between the coil parameter (e.g., clock-wise or counter-clock-wise coil(s)) and the aperture's wall cut. FIGS. 5, 6 and 8 demonstrate apertures that are equal in size, where apertures 51L and 51R are arranged in a clockwise rotation along axis A:A, 81L and 82R are arranged in a counter-clockwise rotation; whereas apertures 61L and 61R are of opposite rotation, so that, for each for each rotation direction, one of the apertures will easily collapse and the other will not. FIG. 7 shows an embodiment where, again, one aperture collapses. In FIG. 7, however, the 9 o'clock aperture is smaller than the 3 o'clock aperture, see 71L and 71R, respectively.

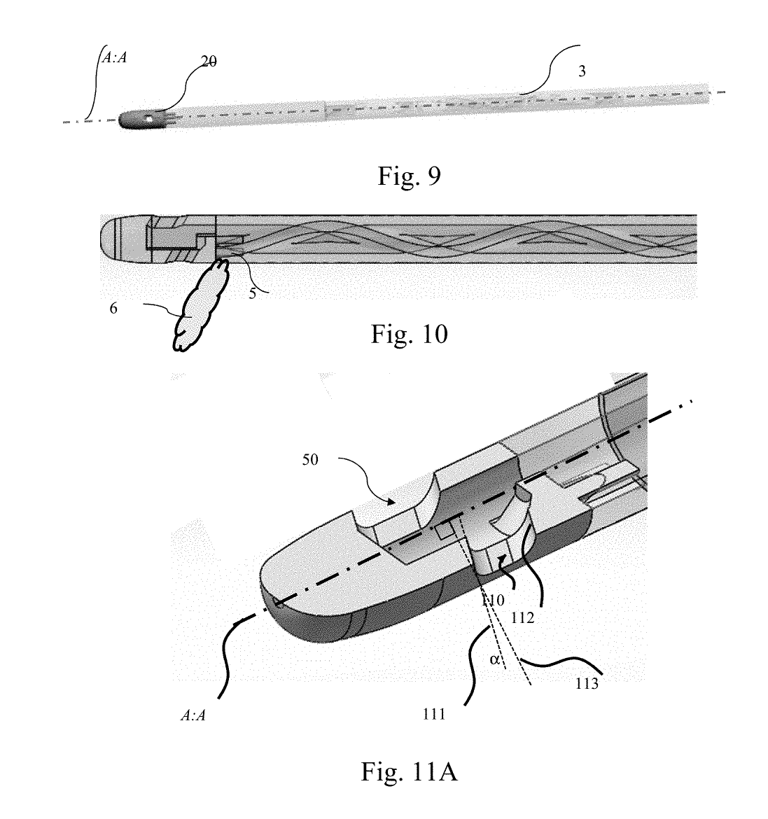

Another aspect of the invention is to disclose a catheter comprising an asymmetric tip (e.g., 20) in combination with either a symmetric or an asymmetric shaft (3), as schematically presented in FIG. 9. The catheter may also comprise other modules, such as a balloon (6), a balloon inflating bore (5) etc., as illustrated in FIG. 10. A three-dimensional in-scale presentation of an asymmetric tip (here 50) is provided in FIG. 11. Another in-scale presentations (40) of an asymmetric tip is provided in FIG. 12, in two and three dimensions, by the various views 40a-40h.

Another aspect of the invention is to disclose asymmetric tips of various shapes and utilizations, as schematically illustrated in an out-of-scale manner in FIGS. 13-20. FIG. 13 shows an asymmetric elastomeric catheterization tip with a hollow interior which has no apertures. FIG. 14 shows an asymmetric elastomeric catheterization tip, where the tip has no apertures and is solid but consists of two materials, an exterior wall material and an interior fill material, with the two materials m=having different properties. The two different materials are asymmetrically disposed within the dome; in FIG. 14, different portions of the exterior wall have different thicknesses. The different materials will differ in at least one material property. The material property difference can be, for example, hardness, Young's modulus, Poisson's ratio and any combination thereof. For example, the two materials can have different hardnesses, with at least one first elastomer being softer (e.g., 30 Shore-A) and at least one second elastomer being harder (e.g., 65 Shore-A material). In some embodiments, the tips comprise more than two materials which differ in at least one property.

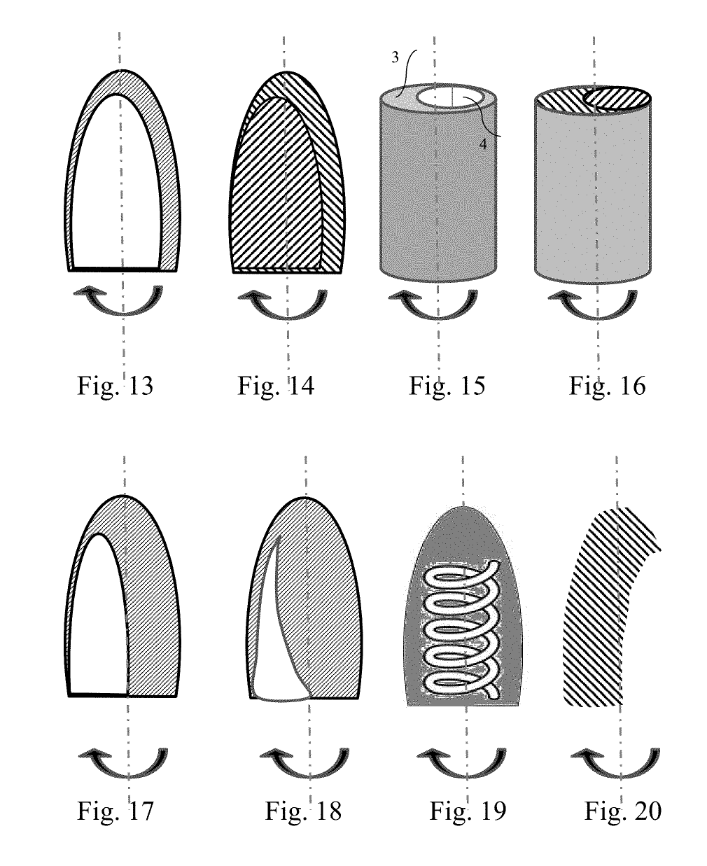

FIGS. 15 and 16 show an embodiment which comprises two different materials where the most distal end of the tip is completely or almost-completely flat.

FIGS. 17 to 20 show cross sections of somewhat different tips, where the asymmetric geometry is enhanced. FIG. 17 discloses a tip with an asymmetric inner envelope, where the inner envelope is symmetrical but its axis is parallel to, but displaced from, the main axis A:A of the tip. In embodiments of this type, the wall thickness varies in the radial direction, giving the desired difference in moment of inertia.

FIG. 18 discloses a tip where the inner envelope is itself asymmetric.

FIG. 19 discloses another embodiment with asymmetric inner envelope. In this exemplary embodiment, the inner envelope forms a coil-like air filled inner section.

FIG. 20 discloses an asymmetric outer envelope. In this exemplary embodiment, the outer envelope forms a collapsed coil-like elastomeric envelope.

Another aspect of the invention is to disclose asymmetric tips as defined and described hereinabove, including combinations of features and characteristics, and including examples as illustrated and explained in any of FIGS. 2 to 20.

The above summary of the various representative embodiments of the invention is not intended to describe each illustrated embodiment or every implementation of the invention. Rather, the embodiments are chosen and described so that others skilled in the art can appreciate and understand the principles and practices of the invention. The figures and the detailed description that follow more particularly exemplify these embodiments.

1. A catheter tip having a main longitudinal axis with a tapered distal portion having an enveloping external wall defining an inner space; wherein the thickness of said wall is radially asymmetric with respect to said axis.

2. A catheter tip having a main longitudinal axis having a tapered distal portion comprising an enveloping external wall defining an inner space; wherein said tip is provided with at least one first small aperture and at least one second large aperture, said apertures are located approximately opposite each other.

3. The catheter tip of claim 1, wherein said tip is provided with at least one first small aperture and at least one second large aperture, said apertures are located approximately opposite each other.

4. A catheter tip having a main longitudinal axis with a tapered distal portion having an enveloping external wall defining an inner space; wherein said tip is provided with at least one first small aperture and at least one second large aperture, said apertures are located approximately opposite each other; and wherein said apertures are cutouts; jambs of said cutouts are tilted from the outer edge to the inner edge away from the perpendicular.

5. The catheter tip of claim 4; wherein the thickness of said wall is radially asymmetric with respect to said axis.

6. The catheter tip of claim 4, wherein said tip is provided with at least one first small aperture and at least one second large aperture, said apertures are located approximately opposite each other.

7. A catheter comprising a tip as defined in any of claims 1 to 6.
